(19) United States
(12) Patent Application Publication
(10) Pub. No.: US 2009/0227853 A1
(43) Pub. Date: Sep. 10, 2009

(54) WEARABLE OPTICAL PULSE PLETHYSMOGRAPHY SENSORS OR PULSE OXIMETRY SENSORS BASED WEARABLE HEART RATE MONITORING SYSTEMS

(76) Inventor: Ravindra Wijesiriwardana, Bentonville, AR (US)

Correspondence Address: RAVINDRA WIJESIRIWARDANA, 2001 LATROBE DRIVE APT 20, BENTONVILLE, AR, 72712, US

(21) Appl. No.: 12/395,791

(22) Filed: Mar. 2, 2009

Related U.S. Application Data
(60) Provisional application No. 61/033,244, filed on Mar. 3, 2008.

(51) Int. Cl. A61B 5/1455

(52) U.S. Cl. 600/324; 600/344; 600/323

(57) ABSTRACT

A new PPG/SpO₂ based ear hook sensor was constructed with the PPG/SpO₂ sensors are attached to the skin in the regions of superficial artery and vein (010) and posterior auricular artery and vein around the ear (011 of FIG. 1C). An ear wearable heart rate monitor is constructed with PPG/SpO₂ sensors are attached to the skin in the regions of superficial artery and vein (010) and posterior auricular artery and vein around the ear (011 of FIG. 1C) (temporal region of the head). This sensor system is less vulnerable to motion artifacts hence capable of producing high quality PPG signals under motion conditions such as running and exercising.

### CROSS-REFERENCE TO RELATED APPLICATIONS

This application claims the benefit of provisional patent application Ser. No. 61/033,244, filed Mar. 3, 2008 by the present inventor.

### FEDERALLY SPONSORED RESEARCH

Not Applicable

### SEQUENCE LISTING OR PROGRAM

Not Applicable

### BACKGROUND-FIELD

This application relates to wearable pulse plethysmography (PPG) sensors or pulse oximetry (SpO₂) sensors based heart rate monitoring systems.

### BACKGROUND-PRIOR ART

Wearable optical pulse plethysmography (PPG)/SpO₂ sensors are used to measure heart rate information. These (PPG)/SpO₂ sensors based heart rate monitoring systems can be divided into several categories depending on the orientation and the location of the sensor. The following categories including ear pinna wearable, in ear wearable, finger wearable, body surface wearable and head wearable can be identified. The heart rate monitoring and the display systems based on these sensors are either worn by the wearer or placed on table top. The present optical PPG/SpO₂ sensor based heart rate monitors are uncomfortable to wear, poor in signal quality, poor in signal accuracy and poor in reliability due to the motion artifacts produced during the motion of the wearer.

The present invention includes a new wearable PPG/SpO₂ senor arrangement and a wearable heart rate monitoring device that can be used to measure and monitor the heart rate reliably and accurately with less motion artifact having better signal to noise ratio.

### SUMMERY OF THE PRESENT INVENTION

An ear wearable hook with a PPG/SpO₂ sensor, the sensor is attached to the skin on the head in the region of superficial artery and vein near the ear (010). In a second approach to increase the signal quality an ear hook is invented with multiple PPG/SpO₂ sensors. The sensors are attached to the skin of the head in the regions of superficial artery and vein (010) and posterior auricular artery and vein around the ear (011 of FIG. 1C). The PPG/SpO₂ sensors are attached to the skin with adhesives. The PPG signal picked up by these sensors are due to the fluctuations of the blood flow in these regions. These sensor units are shown in the FIG. 1A and FIG. 1B. A person wearing the sensor unit is shown in the FIG. 1D. The sensor unit is connected to the signal conditioning device via the conductive cable (004).

In addition an ear wearable heart rate monitoring device is monitored with the PPG/SpO₂ sensor are picking up the fluctuation of the blood flow from the region marked on the FIG. 1C (010,011). The sensory unit is constructed according to the previous chapter and this sensor unit is connected to the ear wearable heart rate monitoring unit (FIG. 2A). A person wearing the unit is shown in the FIG. 2C. In addition PPG/SpO₂ sensors can be connected to the ear wearable heart rate monitoring via a cable. The PPG/SpO₂ sensor is attached to the skin by using adhesives. The ear wearable heart rate monitoring unit has speaker that is capable of audio feed back. The switches (007) on the device are used for controlling the device. This device is capable of wireless PPG signal transmitting and control signals or audio feedback physiological information receiving.

### DRAWING—Figures

FIG. 1A—Shows the ear hook with PPG/SpO₂ sensor located on the hook such that it sets on either the regions 010 or 011.

FIG. 1B—Shows the ear hook with multiple PPG/SpO₂ sensors located on the hook such that they set on the regions 010 and 011.

FIG. 1C—Shows the regions of superficial artery and vein (010) and posterior auricular artery and vein around the ear (011).

FIG. 1D—A person wearing the ear hook PPG/SpO₂ sensor unit.

FIG. 2A—An ear wearable wireless heart rate monitoring unit with ear hook PPG/SpO₂ sensor the unit hook.

FIG. 2B—An ear wearable wireless heart rate monitoring unit with PPG/SpO₂ sensor connected via cable.

FIG. 2C—A person wearing a device described in FIG. 2A.

FIG. 2D—A person wearing a device described in FIG. 2B.

FIG. 3B—PPG signal form a ear pinna connected PPG/SpO₂ sensor when the wearer in moving.

FIG. 3A—PPG signal form the new PPG/SpO₂ sensor hook when the wearer in moving.

### DRAWINGS—Reference Numerals

001—The ear hook
002—PPG/SpO₂ sensor
003—Ear plug of the sensor hook
004—PPG/SpO₂ Sensor/s power and signal pathways.
010—Region of superficial artery and vein near the ear.
011—Region of posterior auricular artery and vein around the ear.
003—Ear plug of the ear wearable heart rate monitor.
005—The housing of the ear wearable heart rate monitor.
006—The connector cable that connects the PPG/SpO₂ sensor/s to the ear wearable heart rate monitor.
007—The switches of the ear wearable heart rate monitor.

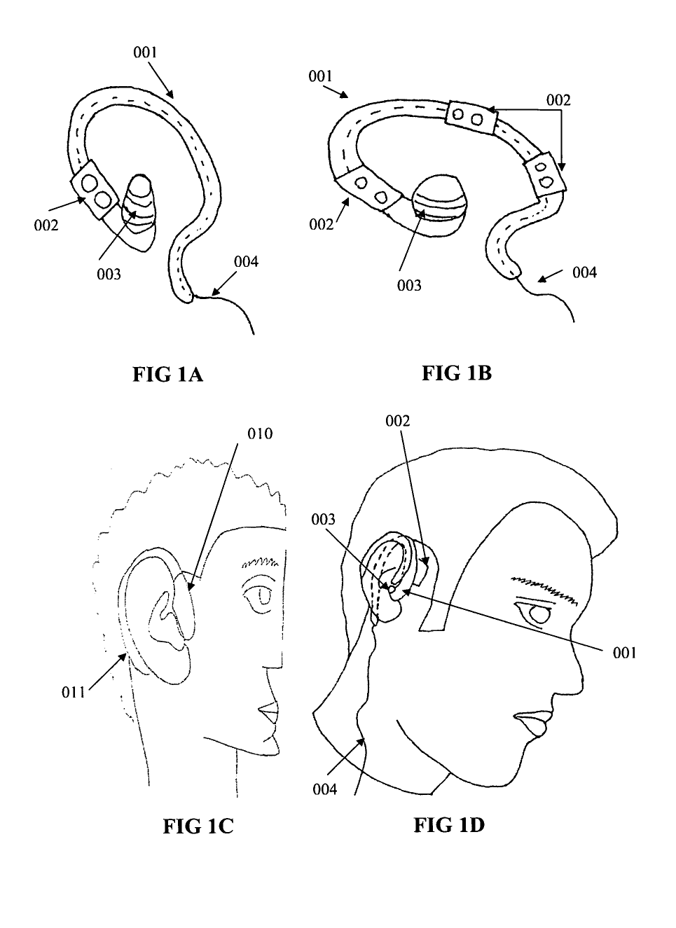

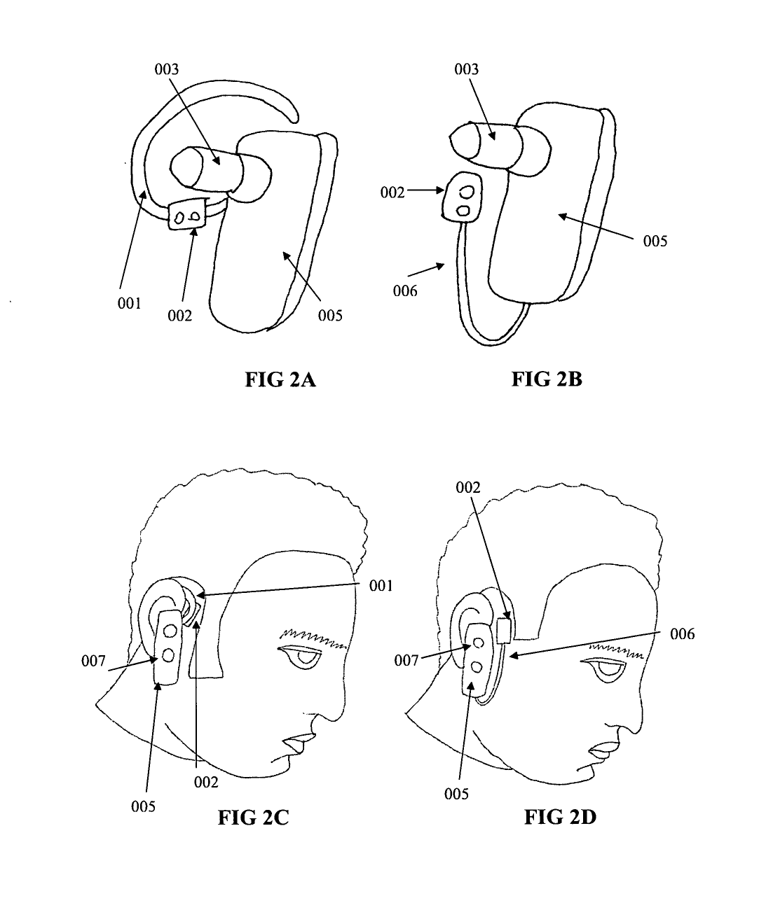

### DETAILED DESCRIPTION of FIG. 1A, FIG. 1B, FIG. 2A, FIG. 2B

FIG. 1A shows the ear hook with PPG/SpO₂ sensor located on the hook such that it sets on either the regions 010 or 011. The ear hook is designed to set around the ear. The PPG/SpO₂ sensor is connected to the ear hook. The signal and power conductive pathways are embedded inside the ear hook. The ear hook with the PPG/SpO₂ sensor consists of an ear plug. This ear plug is made with softer and flexible materials. The signal and power lines of the sensor are connect to the signal conditioning unit via the conduction pathways (004).

FIG. 1B shows the ear hook with multiple PPG/SpO₂ sensors located on the hook such that they set on the regions 010 and 011. This is to increase the reliability and the accuracy of the heart rate information. Two or more PPG/SpO₂ sensors may used for the signal pickup.

FIG. 2A shows an ear wearable wireless heart rate monitoring unit with ear hook PPG/SpO₂ sensor the unit hook . The hook (001) connected to the Housing (005) and housing has an ear plug (003). This unit may contain an audio feedback unit. The unit is capable of wireless information transmission.

FIG. 2B shows an ear wearable wireless heart rate monitoring unit with PPG/SpO₂ sensor connected via a cable. In this arrangement the stress on the ear is lower while one wearing the device.

### Operation of the System

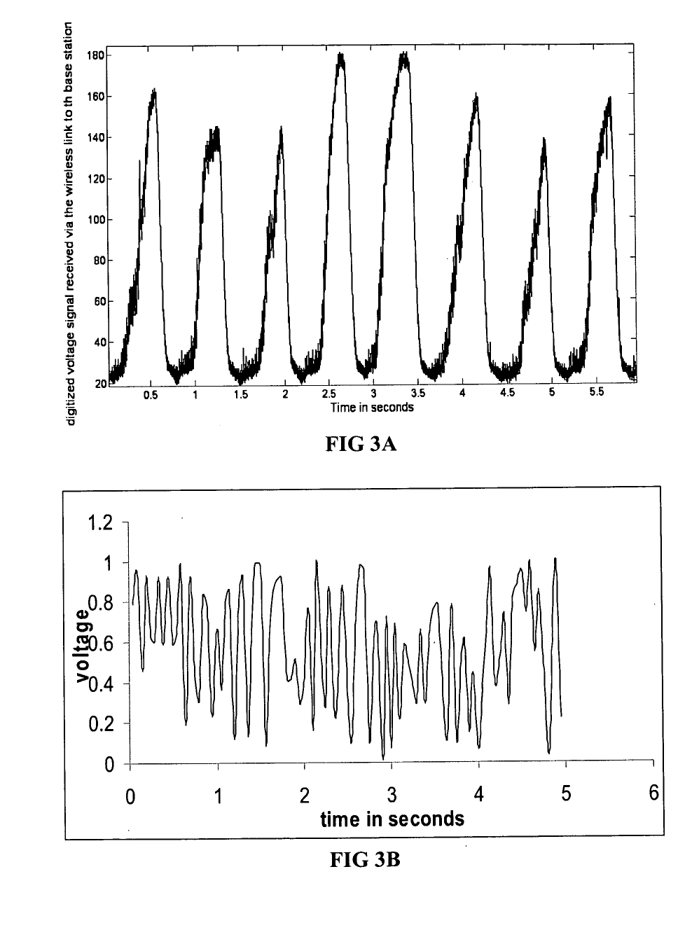

Upon wearing the ear hook sensor with single or multiple PPG/SpO₂ sensors, the PPG/SpO₂ sensors sticks to the skin via adhesives. Since the pulse is stronger in the area of the sensors low power is required to detect the pulse. The PPG signals are picked up by the sensors and sent to the monitoring unit via the wireless link. This new innovative system is tested against the ear wearable PPG/SpO₂ sensors based heart rate monitoring unit where the PPG/SpO₂ sensors are connect to the ear pinna. The results are shown in the FIG. 3A and FIG. 3B. FIG. 3A shows the PPG signal of the present invention and FIG. 3B shows the PPG signal pick up by the PPG/SpO₂ sensors based heart rate monitoring unit where the PPG/SpO₂ sensors are connect to the ear pinna. These test results were recorded under the same motion level while the wearer was running at 5 mph. It is clear from these results that the present invention is much better under motion conditions than the existing PPG/SpO₂ sensors based heart rate monitoring unit where the PPG/SpO₂ sensors are connect to the ear pinna.

I claim:

1. An optical PPG/SpO₂ sensor and sensor embodiment comprise of;
(a) An ear hook for mounting the sensor;
(b) An ear plug connected to the ear hook so that the hook can be connected well to the wearer;
(c) At least one PPG/SpO₂ sensor or PPG/SpO₂ sensors of the embodiment attached to the temple region of the head of the wearer;
(d) Electrical signals and power pathways to the sensors from a control unit.

2. A device according to claim 1 where the sensors are used for monitoring heart rate and blood gas concentrations.

3. An ear wearable wireless physiological information monitoring system comprising sensors of claim 1.

4. A wearable wireless physiological information monitoring system connected to a device according to claim 1, which can be embedded or attached to following wearable devices including: jackets, head bands, a cap, a hat, a helmet, a swimming cap, a pair of optical glasses, goggles, a shirt, a chest strap or a trouser.

5. Use of devices according to claim 3 and claim 4 to monitor the heart rate, respiration information or blood gas concentration of the wearer.

6. A device according to claim 3 or claim 4 having a speaker for the audio feed back of the physiological information.

7. A sensor recording to claim 1 having a speaker for the audio feed back to the wearer.

8. A sensor according to claim 1 or claim 7 having a temperature sensor in the ear plug.

9. A wrist wearable display, PDA, smart phone or external base station for displaying, recording, processing and analyzing the information from the devices in claim 3 and claim 4.

10. A device according to claim 3 or claim 4 capable of transmitting information via public data networks to a remote station for display, analyzing, recording and decision making.

11. A device according to claim 3 or claim 4 having control press button switches for the controlling of the system.

12. A device according to claim 1 or claim 8 where the adhesion of the sensors to the skin is done by using transparent double side sticky tape or sticky pads or with double side sticky tape or sticky pads with a hole for the optical sensor area.

13. A device according to claim 3 having a sensor unit according claim 1 or claim 8 on one ear, the wireless signal conditioning and control unit on the other ear and an electrical conduction cables connecting them.
